(19) United States
(12) Patent Application Publication
(10) Pub. No.: US 2005/0159708 A1
(43) Pub. Date: Jul. 21, 2005

(54) Infusion pump, control program, semiconductor means and method for the dosed administration of a medicinal liquid

(76) Inventor: Rudolf Sidler, Langendorf (CH)

Correspondence Address: DORSEY & WHITNEY LLP;INTELLECTUAL PROPERTY DEPARTMENT, 50 SOUTH SIXTH STREET, MINNEAPOLIS, MN, 55402-1498, US

(21) Appl. No.: 11/041,513

(22) Filed: Jan. 24, 2005

Related U.S. Application Data
(63) Continuation of application No. PCT/CH03/00456, filed on Jul. 8, 2003.

(30) Foreign Application Priority Data
Jul. 24, 2002 (DE) 102 33 622.9

(51) Int. Cl. A61M 37/00

(52) U.S. Cl. 604/132; 700/282

(57) ABSTRACT

An infusion pump for the dosed administration of a liquid includes a liquid conveying device, for example a pressure spring permanently impinging a piston tappet with pressure, and a distribution control device, for example a stop valve, for dosing the liquid distribution. The liquid conveying device impinges the liquid at the inlet of the distribution control device with pressure and the liquid conveying means is decoupled from the distribution control device. Exactness of dosage thus depends on the work tolerances of the liquid conveying means and is substantially only controlled by the exactness of the distribution control device which can be suitably adjusted. The invention encompasses a control method, a control program and a micro-processor component, and is suitable for mobile infusion pump systems, for example in the long-term administration of micro-doses of insulin to diabetes patients.

### CROSS REFERENCE TO RELATED APPLICATION(S)

This application is a continuation of International Patent Application No. PCT PCT/CH03/00456, filed on Jul. 8, 2003, which claims priority to German Application No. DE 102 33 622.9, filed on Jul. 24, 2002, the contents of which are incorporated their entirety by reference herein.

### BACKGROUND

The invention relates to an infusion pump for administering an injectable medicinal liquid in metered doses, in particular for obtaining a controlled administration of micro-doses of a liquid containing a medicinal or therapeutic substance for a longer period of time, for example for administering insulin or the like over a long period of time. The invention further relates to a method of controlling such administration in metered doses, a control program therefor and a semiconductor component, incorporating such a control program for use in an infusion pump.

Infusion devices are known from the prior art as a means of administering a liquid containing a medicinal or therapeutic substance for a longer period of time by injection into a body volume, for example veins or tissue. An important parameter in this respect is the quantity of liquid to be administered per unit of time, from which the administered substance can be calculated. In hospitals, it is common practice to use infusion bottles suspended above the injection point, from which the liquid is dispensed by force of gravity. Often, a variable hose clamp serves as a dispensing control device. Infusion bottles are not suitable for use as mobile infusion devices or for dispensing rather smaller doses over a longer period of time.

Other infusion pumps are known from the prior art, whereby the operating mode is controlled so that the quantity of liquid administered corresponds to the desired dose. Such infusion pumps have a medicament container, from which the liquid is dispensed by driving a plunger stopper forward. Metering is controlled by the forward drive of the plunger. A reduction gear, for example a toothed rack, may be provided as a means of driving the plunger stopper forwards in order to administer relatively small doses over a long period of time. What such infusion pumps have in common is that the metering accuracy is predetermined by manufacturing tolerances of the infusion pump itself. This makes it necessary to conform to narrow manufacturing tolerances, as a result of which such infusion pumps are usually suitable for use with only one type of medicament container, for example an ampoule.

U.S. Pat. No. 6,348,043 discloses an infusion device in which a medicinal liquid is dispensed from an ampoule by driving forward a plunger stopper serving as a liquid delivery means. Pressure is applied to a membrane on the end face of the plunger stopper by means of a compression spring so that the liquid is dispensed slowly in conjunction with the forward motion of the plunger stopper. A step element serving as a stop is used to predetermine the metered dose and must be precisely manufactured in order to administer relatively small doses. With this device and with the infusion pumps mentioned above, the functions of liquid ejection based on the forward driving motion of the plunger and dose metering are not decoupled from one another, which is what gives rise to said restrictions in terms of metering accuracy and flexibility.

### SUMMARY

An object of the invention is to propose an infusion pump by means of which an accurate and flexible dose of the liquid to be injected can be metered in a simple manner. In particular, the object is to propose a mobile infusion pump for administering relatively small doses of a liquid to be injected over a long period of time. A control method, a control program and a semiconductor component incorporating such a control program are provided for this purpose.

This object is achieved by means of an infusion pump, a control program, a semiconductor component for storing the control program, and a control method.

An infusion pump in accordance with the present invention has a liquid delivery means for delivering the liquid from a liquid container containing the liquid to be injected, as well as a dispensing control device for metering the process of dispensing the liquid. For the purpose of the invention, the liquid delivery means delivers the liquid to an inlet of the dispensing control device by means of pressure, at least immediately before and during the dispensing process, and the liquid delivery means does not interact with the dispensing control device. The dispensing control device is connected downstream of the liquid delivery means in the flow path of the liquid. Both the liquid delivery means and the dispensing control device act on the liquid, the latter downstream of the delivery means. Advantageously, there is no mechanical interaction between the delivery means and the dispensing control device as a means of controlling or regulating the delivery rate as a function of the metered dose.

Accordingly, for the purpose of the invention, the liquid is delivered exclusively by the liquid delivery means, for example by ejection from an ampoule, but it is not used to control the dispensing process or to dispense the liquid in metered doses. The advantage of this is that the liquid delivery means can be made simple and inexpensive because the metering accuracy is not determined by the delivery means, but, in one embodiment, solely or substantially only by the dispensing control device. Advantageously, there is no need to use mechanical precision components for the delivery means. The liquid delivery means can also be made to a more flexible design overall, for example so that it can be used with different types of liquid containers, because the delivery means, for example a threaded rod drive for a plunger stopper, and liquid container, for example an ampoule with such a stopper, no longer have to be designed to co-operate in a precise manner.

For the purpose of the invention, the quantity of liquid dispensed, which is delivered by the liquid delivery means to the dispensing control device by applying pressure, is controlled by the dispensing control device. The advantage of this is that, because the delivery means and dispensing control device do not interact, the latter can be used for different types of delivery means and/or liquid containers, which further enhances the flexibility of the infusion pump.

The fact that there is no interaction between the functions of generating the delivery pressure and metering (metering by the dispensing control device takes place downstream, in the flow direction, of the point at which the delivery pressure is generated) means that reliability is increased in terms of ensuring that only the desired quantity of liquid is effectively dispensed. In the case of conventional infusion pumps in which the liquid delivery means simultaneously assumes the metering function, dispensing may not be correct due to heat expansion of the liquid. In the case of the infusion pump in accordance with the present invention, on the other hand, in which the dispensing control device operates downstream, the pressure upstream of the inlet of the dispensing control device increases in the event of heat expansion. The delivery means may optionally ease off, thereby compensating for all or some of the increase in pressure. Only a volume of liquid downstream of the inlet of the dispensing control device can lead to faulty dispensing if it expands to due heat but this is minimised accordingly, due to the smaller volume compared with conventional infusion pumps. The invention therefore reduces faulty dispensing incurred by temperature fluctuations.

The liquid delivery means delivers the liquid at the inlet to the dispensing control device, at least immediately before and during dispensing of the liquid by the dispensing control device. In one simple, advantageous embodiment, the liquid is delivered to the inlet of the dispensing control device by a permanently applied pressure, thereby obviating the need for additional control means to control the pressure applied.

Preferably, in some embodiments, the dispensing control device temporarily opens an outlet communicating with its inlet so that pressurized liquid can be dispensed in a controlled manner during a pre-settable time. The dispensing control device is preferably controlled by an electronic control means, in order to control the timing, which varies as a function of the dose to be obtained.

In a preferred embodiment, a liquid container provided as a means of accommodating the liquid is connected to the infusion pump or is even integrated in it, thereby obtaining a mobile infusion pump. In this embodiment, the liquid delivery means applies pressure to the liquid accommodated in the liquid container and more particularly preferably does so on a permanent basis, in order to deliver the liquid from a dispensing orifice of the liquid container to the inlet of the dispensing control device. The liquid container is preferably connected to the dispensing control device via a relatively short and not very flexible or inflexible line, so that the pressure causes only negligible deformation of the walls of the line. The dispensing control device may also be provided directly adjoining or in the dispensing orifice of the liquid container and may be integrated in it, for example.

In one preferred embodiment, the liquid delivery means applies pressure to the liquid container permanently so that the liquid is delivered to the dispensing control device in an uncontrolled manner and it is exclusively the dispensing control device which controls dispensing, for which purpose it does not interact with the liquid delivery means.

The liquid container maybe a standard glass or plastic ampoule with a substantially cylindrical cross-section, for example, or it may have an essentially rectangular body, incorporating a plunger stopper which dispenses the liquid from the container when the plunger stopper is driven forward by the liquid delivery means. The liquid container may also be a bag or similar, the wall of which is deformed in order to dispense liquid when pressure is applied. The dispensing orifice is preferably disposed at an end of the liquid container lying essentially opposite the liquid delivery means so that advantageously low forces are sufficient to dispense the liquid.

In one preferred embodiment, the liquid delivery means preferably has a drive means which applies a force to drive the plunger stopper to the dispensing orifice. The drive means is preferably elastically biased and applies a driving elastic force permanently to the plunger stopper in order to drive it forward, for example by means of a forward-biased compression spring, a helical spring mechanism, a rotating spring mechanism, an elastomer compressed in an initial position or a pressurized gas reservoir, for example an inflatable pneumatic system. As long as the dispensing control device assumes a closed position, i.e., a locked position, in which the liquid is prevented from being dispensed, the plunger stopper can not be driven forward because the liquid contained in the liquid container is essentially incompressible. However, once the dispensing control device permits liquid to be dispensed, the through-flow rate is essentially predetermined by the preferably pre-settable elastic force of the drive means and the degree to which the plunger stopper is driven forward as a result, in combination with the size of the flow cross-section and/or the time during which the flow cross-section of the dispensing control device is open, thereby enabling the dose effectively administered to be calculated in a simple manner, taking account of the concentration of medicinal or therapeutic substance contained in the liquid. This calculation function may be operated with the aid of a standard electronic control means, for example any suitable micro-processor, which is advantageously an integral part of the infusion pump.

The liquid delivery means may also be one which applies pressure to the liquid at the inlet of the dispensing control device on an intermittent basis only, in other words before and during operation of the dispensing control device, thereby advantageously reducing the power consumption of the infusion pump. An embodiment of this type is of particular advantage in situations where a motor-driven liquid delivery means is used, for example a motor-driven plunger stopper drive mechanism.

By preference, the pressure at which the liquid is delivered to the inlet of the dispensing control device may be varied, which will lead to a change in the flow rate and hence the actual dose administered, in a known manner.

In all of the embodiments described above, the dispensing control device is preferably permanently closed and its outlet is only temporarily released in order to dispense a metered quantity of liquid. The cross-section of the outlet of the dispensing control device is preferably such that it can be variably pre-set. A control means is preferably provided for controlling the dispensing control device, for example a micro-processor or ASIC.

As a result of another aspect of the invention, a method is also proposed as a means of controlling administration of a liquid to be injected in metered doses, preferably a liquid containing a medicinal or therapeutic substance. In accordance with the method, the liquid is delivered to the inlet of the dispensing control device by or under pressure. The dispensing control device is operated, causing the pressurized liquid to be dispensed, and there is no interaction between the process of pressurizing the liquid and operation of the dispensing control device. The control method proposed by the invention may be used universally for different liquid or medicament containers, liquid delivery means and dispensing control devices. Key control parameters, such as the time during which liquid dispensing is permitted by the dispensing control device, the cross-section of the outlet of the dispensing control device during liquid dispensing, the concentration of the medicinal or therapeutic substance contained in the liquid, the over-pressure prevailing at the dispensing control device, etc., may be entered at the control means, for example by means of a fixed program or by means of sensors provided in the infusion pump, so that precise metering can be achieved by the control method, including over longer periods of time, whilst using a simple and relatively inexpensive infusion pump.

In one embodiment, the control method comprises a control program which is machine-driven or can be read by a microprocessor and is preferably stored on a semiconductor component which can be obtained as a separate component, for example an EPROM, an EEPROM or other suitable component.

Other objects, advantages and features of the present invention will become clear with reference to the following description, the accompanying drawings and the appended claims.

### BRIEF DESCRIPTION OF THE DRAWINGS

FIG. 1 is a schematic diagram illustrating the structure of an infusion pump in accordance with the present invention;

FIG. 2 is a schematic diagram illustrating the structure of an infusion pump in accordance with the present invention in which a peristaltic pump is used as the dispensing control device; and

FIG. 3 is a schematic diagram illustrating a cycle of the peristaltic pump illustrated in FIG. 2 for controlling and metering the quantity of liquid dispensed.

### DETAILED DESCRIPTION OF THE DRAWINGS

In the following description, the same reference numbers are used in the drawings to denote identical or similarly acting components and functional groups.

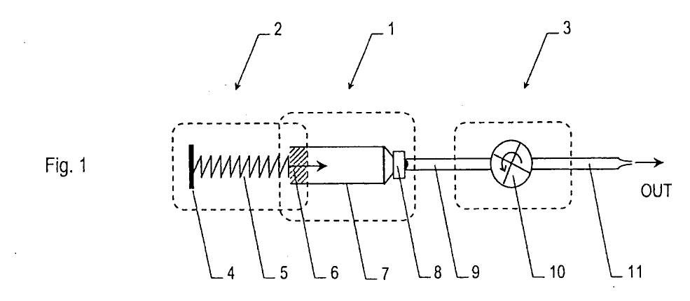

FIG. 1 is a schematic illustration of the structure of an embodiment of a non-interacting infusion pump in accordance with the present invention. The infusion pump comprises a medicament container 1, a liquid delivery means 2, by means of which a delivery pressure is generated in the container 1, and a dispensing control device 3, which is disposed downstream of the medicament container 1 in order to control the liquid dispensed from the medicament container 1. The medicament container 1 has an essentially cylindrical or polygonal, for example rectangular, wall 7, at the proximal end of which a plunger stopper 6 is inserted, which, when driven axially forwards, delivers the liquid containing a medicinal or therapeutic substance accommodated in the medicament container 1 through a dispensing orifice 8 into the connecting line 9 in or to the dispensing control device 3. The medicament container 1 may also be a bag with at least one flexible side wall, which is compressed in order to dispense the liquid contained in the bag, for example by a motor-driven or electromagnetically driven mechanism, which is able to push a side wall of the bag inwards.

The pressure-generating means 2 is a drive means 5, for example a compression spring biased to a maximum when the infusion pump is in the initial state, i.e., with the plunger stopper 6 in the proximal end position, a biased helical spring mechanism which permanently applies pressure to the plunger stopper 6, a pre-tensed rotating spring mechanism similar to that used for a clock mechanism which drives the plunger stopper 6 forward, an elastomer which is biased in the initial state or a pneumatic system which is inflated to its maximum when the infusion pump is in its initial state. The drive means 5 exerts a driving force which acts on the plunger stopper 6 in an axial direction towards the dispensing orifice 8, which in the embodiment illustrated here is an elastic force. The connecting line 9 and the side wall 7 of the medicament container 1 are preferably inflexible. The incompressible liquid contained in the medicament container 1 and the connecting line 9 can not be dispensed by the plunger stopper 6 through an outlet 11 of the dispensing control device 3 except when the outlet 11 of the dispensing control device 3 is opened. In the case of the embodiment illustrated in FIG. 1, in which a not necessarily constant over-pressure permanently prevails at the inlet of the dispensing control device 3 connected to the container 1, the liquid delivery means 2 and the dispensing control device 3 are decoupled from one another. The dispensing control device 3 and liquid delivery means 2 may be operated, manipulated or replaced independently of one another without essentially affecting the operating mode of the infusion pump.

The dispensing control device 3 is used exclusively as a means of dispensing the liquid in metered doses and does not in any way affect the liquid delivery and pressure-generating means 2.

The infusion pump illustrated in FIG. 1 is distinctive due to a modular structure, in which the most important individual components, such as the dispensing control device 3, medicament container 1 and liquid delivery means 2, can be readily exchanged and replaced by other components, permitting adaptation to specific applications, for example. In particular, the medicament container 1 may be replaced by another of a different size or different shape, etc., provided the liquid delivery means 2 is capable of performing the intended function, for example is able to apply pressure to the plunger stopper 6 permanently or at least immediately before and after the outlet 11 of the dispensing control device 3 is opened. To this end, a universal attachment point may be provided on the rear face of the plunger stopper 6 to enable the plunger stopper 6 to be connected to the drive means 5, for example a universal thread for selectively connecting different drive elements 5, for example a compression spring, a rotating spring mechanism, a helical spring mechanism, a pre-biased elastomer, a pneumatic chamber or a threaded rod of a conventional motor-driven plunger stopper drive.

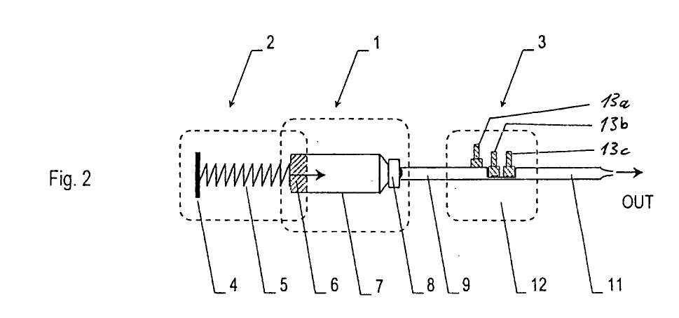

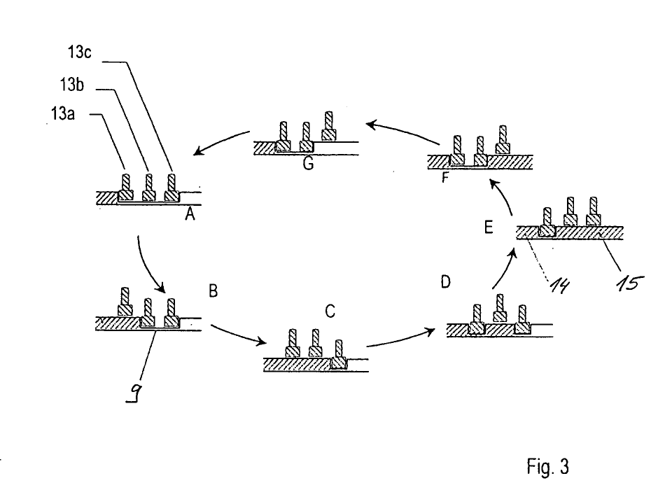

Any device which allows through only an exactly defined quantity of liquid in spite of a permanent and/or variable over-pressure at its inlet may used as the dispensing control device 3. Examples of dispensing control devices 3 are a shut-off valve which meters the dispensed quantity of liquid over time in conjunction with a component with a defined flow resistance whenever the shut-off valve is opened. Another example is a circulation pump which is tightly sealed in the non-operating state, for example the peristaltic pump illustrated in FIGS. 2 and 3. Another example is a modified carousel or squeegee pump which permanently and fully squeezes a hose so that no liquid can be dispensed and which squeezes the hose less hard to permit dispensing so that a pre-settable hose cross-section is released in order to dispense liquid. Another example is a circulation pump that is not tightly sealed in the non-operating state, for example a diffuser pump, which co-operates with a valve disposed upstream or downstream of the circulation pump which permanently blocks an outlet in the non-operating state and is not opened except under the action of the liquid dispensing system.

The dispensing control device 3 therefore is essentially different in terms of a passive operating mode, whereby pressure is applied permanently or quasi continuously to the inlet of the dispensing control device 3 and a closure means such as a valve, a nozzle or a reduction in cross-section, for example, is opened on a timed basis in order to dispense liquid, and an active operating mode whereby the liquid delivery means merely ensures that the liquid to be administered is delivered in a sufficient quantity and the task of dispensing is actively assumed by the dispensing control device 3, for example by means of a peristaltic pump as described in connection with FIGS. 2 and 3, a carousel pump or similar, which delivers a pre-settable quantity of liquid through the outlet 11.

As a result of the invention, the metering accuracy is not restricted by the liquid delivery means 2 and instead, the metering accuracy is pre-determined by the dispensing control device 3. This means that a simple and inexpensive liquid delivery means 2 with broad manufacturing tolerances may be used. The infusion pump in accordance with the invention may, therefore, have an ejection mechanism of a relatively small design, for which manufacturing tolerances play an even greater role. In particular, when manufacturing the ejection mechanism, there is no need to use mechanical precision elements, for example precise threaded rods and similar.

In order to control the dispensing control device 3, a control program may be provided, for example integrated in a semiconductor component, in which key parameters of said infusion pump can be stored or entered and the dispensing control device 3 controlled in an appropriate manner in order to achieve the desired dosage. Key control parameters specifically include the driving force by which the plunger stopper 6 is pressurized to drive it forward, either permanently or at least when the liquid is being dispensed, the cross-section of the connecting line 9, the cross-section of the outlet 11 and any other components connected downstream but not illustrated, such as valves or conduits and the like, as well as the flow cross-section of the outlet 11 of the dispensing control device 3. The driving force by which the plunger 6 is pressurized can be used to calculate the over-pressure prevailing at the inlet of the dispensing control device 3, from which the achievable flow rate can be calculated in a known manner, given that the flow cross-sections of the elements determining the liquid flow are known. Knowing the concentration of the medicinal or therapeutic substance contained in the liquid, the flow rate can in turn be used to calculate the dose of substance actually administered. Having studied the description given above, appropriate computing and control algorithms will immediately be evident to the person skilled in the art and these therefore require no further explanation.

Key parameters, and operational parameters, for the control program, for example the driving force by means of which the plunger stopper 6 is pressurized, the over-pressure prevailing at the inlet of the dispensing control device 3, etc., may be determined by means of a sensor or sensors, known to the skilled person, disposed at appropriate points in the infusion pump, with their output or readings being linked, communicated or forwarded to the control program as the basis for calculating an appropriate timing for controlling the dispensing control device 3.

An infusion pump in accordance with the present invention is suitable for dispensing relatively small doses over a long period of time for the permanent treatment of illnesses, for example for adjusting the blood sugar of diabetic patients. Depending on the control program, to which measurement signals of a sensor for measuring blood sugar levels may be forwarded, the times and doses of insulin stored in the medicament container 1 to be administered can be pre-set.

In embodiments in which the plunger stopper 6 is permanently mechanically biased, an infusion pump that is particularly economical on energy can be achieved because the only power consumers are essentially the dispensing control device 3 and its control program. The drive means 5, for example a return spring, can be mechanically pre-biased, for example by hand, before initiating operation of the infusion pump. Whilst the infusion pump is operating, the drive means 5 eases off its biasing force, either totally or preferably to a certain degree only, as the driving force is generated when the plunger stopper 6 is driven forward.

FIG. 2 illustrates a variant of the embodiment illustrated in FIG. 1, in which the dispensing control device 3 is provided in the form of a peristaltic pump 12. The peristaltic pump 12 has three fingers 13a, 13b and 13c, which can be driven by means of cams and a rotation shaft or other suitable means (not illustrated) in order to effect a movement substantially perpendicular to the flow direction between a top position, in which a cross-section of the connecting line 9 is released, and a bottom position in which the cross-section of the connecting line 9 is preferably completely blocked, so that no liquid can be fed to the outlet 11 of the peristaltic pump 12.

The peristaltic pump 12 performs the cycle schematically illustrated in FIG. 3, which starts with the initial state A in which all fingers 13a-c block the cross-section of the connecting line 9 so that the volume of liquid 14 upstream is blocked upstream of the rear finger 13a. The rear finger 13a is then raised to the top end position, so that the upstream volume of liquid 14 advances as far as the middle finger 13b. When the middle finger 13b is raised, the upstream volume of liquid 14 is conveyed on as far as the downstream front finger 13c. The rear finger 13a is then moved into the bottom end position so that a part of the upstream volume of liquid 14 is enclosed between the front finger 13a and the rear finger 13c. By selecting the stroke height of the middle finger 13b, the actual administered dose can be pre-set if the middle finger 13b is also able to assume and retain intermediate positions in addition to its two end positions. The initial state A is resumed when the front finger 13a is opened (step E) and the middle finger 13b is transferred into the bottom end position (step F) and the rear finger 13c transferred to the bottom end position (step F), in which a quantity of liquid predetermined by the dispensing control device 3 was dispensed to the downstream volume of liquid 15.

In this variant, the upstream volume of liquid 14 is conveyed into the peristaltic pump 12 by means of the over-pressure prevailing at the inlet of the peristaltic pump 12, from where the liquid is dispensed in a controlled manner under the control of said control program.

By varying the control parameters of the control program, the liquid can be dispensed on a variable basis and adjusted to handle a plurality of different components of the infusion pump, as will be immediately evident to the skilled person on studying this description. The liquid may be dispensed on the basis of a pre-programmed pattern, which may be input by an officiating doctor and/or in accordance with the wishes of the patient, for example. The infusion pump may be operated with a replaceable medicament container. Of particular advantage is the fact that the infusion pump described above can be used as a modular infusion pump system, whereby an identical dispensing control device can be used for all medicament modules, i.e., comprising ejection mechanism and medicament container.

In the foregoing description, embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An infusion pump for administering metered doses of a medicinal liquid to be injected, comprising:
a liquid delivery means for delivering the liquid from a liquid container containing the liquid to be injected; and
a dispensing control device for metering dispensing of the liquid, wherein the liquid delivery means delivers the liquid to an inlet of the dispensing control device by pressure and the liquid delivery means does not interact with the dispensing control device.

2. The infusion pump as claimed in claim 1, wherein the liquid delivery means permanently delivers the liquid to the inlet of the dispensing control device by pressure.

3. The infusion pump as claimed in claim 1, further comprising a liquid container for accommodating the liquid, wherein the liquid delivery means applies pressure to the liquid accommodated in the liquid container so that the liquid is delivered from a dispensing orifice of the liquid container to the inlet of the dispensing control device.

4. The infusion pump as claimed in claim 2, in which the liquid delivery means permanently applies pressure to the liquid container so that the liquid is delivered to the dispensing control device in an uncontrolled manner.

5. The infusion pump as claimed in claim 3, in which the liquid delivery means applies pressure to the liquid container at an end of the liquid container lying opposite the dispensing orifice.

6. The infusion pump as claimed in claim 3, in which the liquid container has a plunger stopper which dispenses the liquid as it is driven, the liquid delivery means driving the plunger stopper forward.

7. The infusion pump as claimed in claim 6, in which the liquid delivery means has a drive means in order to apply force to the plunger stopper to drive it as far as the dispensing orifice.

8. The infusion pump as claimed in claim 7, in which the drive means is a motor-driven plunger stopper drive having a controlled impact force for driving the plunger stopper forwards.

9. The infusion pump as claimed in claim 7, in which the drive means is one of a helical spring mechanism, a rotating spring mechanism, an elastomer, a capped pressurized gas reservoir, or a pneumatic system.

10. The infusion pump as claimed in claim 1, in which the liquid delivery means is timed in order to apply pressure to the inlet of the dispensing control device, at least before and during operation of the dispensing control device, for dispensing purposes.

11. The infusion pump as claimed in claim 1, in which the liquid is delivered to the inlet of the dispensing control device at a variable pressure.

12. The infusion pump as claimed in claim 1, in which the dispensing control device closes in a non-operating state and the pump further comprises a control means to dispense a metered quantity of liquid.

13. The infusion pump as claimed in claim 1, in which the dispensing control device comprises a shut-off valve and a component with a defined flow resistance whereby a metered quantity of liquid is dispensed during the time the valve is open.

14. The infusion pump as claimed in claim 1, in which the dispensing control device comprises a tightly sealed circulation pump in the non-operating state.

15. The infusion pump as claimed in claim 14, in which the dispensing control device comprises a peristaltic pump.

16. The infusion pump as claimed in claim 14, in which the dispensing control device comprises one of a carousel or squeegee pump.

17. The infusion pump as claimed in claim 1, in which the dispensing control device comprises a circulation pump that is not tightly sealed in the non-operating state.

18. The infusion pump as claimed in claim 17, in which the dispensing control device comprises a diffusion pump with a shut-off valve disposed downstream.

19. A method of dispensing of a medicinal liquid, comprising the steps of:
delivering the liquid to an inlet of a dispensing control device by means of pressure; and
operating the dispensing control device, causing the pressurized liquid to be dispensed, wherein the pressurization of the liquid for delivery is operationally isolated from the operation of the dispensing control device.

20. A method of controlling the dispensing of a medicinal liquid, comprising the steps of:
delivering the liquid to an inlet of a dispensing control device by means of pressure; and
operating the dispensing control device, causing the pressurized liquid to be dispensed, wherein pressurization of the liquid is decoupled from the operation of the dispensing control device.

21. A control program for an infusion pump implementing a method comprising the steps of delivering a liquid to an inlet of a dispensing control device by means of pressure, and operating the dispensing control device, causing the pressurized liquid to be dispensed, whereby pressurization of the liquid is decoupled from the operation of the dispensing control device, said program comprising program code means which prompt implementation of said delivering and operating when run on a control means operably coupled to the infusion pump.

22. The control program according to claim 21, wherein the program is stored in a semiconductor component operably associated with the infusion pump.
